(19) United States
(12) Patent Application Publication
(10) Pub. No.: US 2011/0166629 A1
(43) Pub. Date: Jul. 7, 2011

(54) MEASURING ANTENNA MATCHING VIA TRANSMITTER CURRENT DRAW

(76) Inventors: Philip G. Dion, Blaine, MN (US); Dennis E. Larson, White Bear Lake Township, MN (US); Sasidhar Vajha, Brooklyn Park, MN (US)

(21) Appl. No.: 12/977,166

(22) Filed: Dec. 23, 2010

Related U.S. Application Data
(60) Provisional application No. 61/292,323, filed on Jan. 5, 2010.

(51) Int. Cl. A61N 1/08; H04B 1/40

(52) U.S. Cl. 607/60; 455/90.1

(57) ABSTRACT

This document discusses, among other things, an implantable telemetry system including an RF transceiver configured to drive an antenna and a matching circuit, configured to be coupled to the antenna, the matching circuit including a control parameter configured to adjust the impedance of the matching circuit. The implantable telemetry system also includes a control circuit configured to receive an indication of the RF transceiver current draw and to control the control parameter of the matching circuit using the received indication of current draw. In an example, the control parameter is controlled to decrease the RF transceiver current draw.

### CROSS-REFERENCE TO RELATED APPLICATIONS

This application claims the benefit of U.S. Provisional Application No. 61/292,323, filed on Jan. 5, 2010, under 35 U.S.C. §119(e), which is incorporated herein by reference in its entirety.

### BACKGROUND

Medical devices can be implanted in a body to perform tasks including monitoring, detecting, or sensing physiological information in or otherwise associated with the body, diagnosing a physiological condition or disease, treating or providing a therapy for a physiological condition or disease, or restoring or otherwise altering the function of an organ or a tissue. Examples of an implantable medical device can include a cardiac rhythm management device, such as a pacemaker, a cardiac resynchronization therapy device, a cardioverter or defibrillator, a neurological stimulator, a neuromuscular stimulator, or a drug delivery system. In certain examples, the implantable medical device can include a radio frequency (RF) transceiver and an antenna, coupled to the RF transceiver, the combination of which can be configured to provide wireless communication between the implantable medical device and an external device, e.g., to send information (such as physiological or other information) from the implantable medical device to the external device, or to receive information (e.g., such as programming instructions) at the implantable medical device from the external device.

Magnetic coupling can be used to provide short-range (e.g., a few centimeters) communication between an implantable medical device implanted in a body and an external device, or between an implantable medical device outside of the body and an external device. However, magnetic coupling communication largely relies on near-field radiation, where the field distribution is highly dependent upon the distance from, and orientation of, the antenna, which limits the effective range of wireless communication between the implantable medical device and the external device.

As an alternative to magnetic coupling, or in addition to magnetic coupling, low power RF communication, having an extended range over magnetic coupling, can be used to provide communication between an implantable medical device and an external device.

### OVERVIEW

In many situations, an RF transceiver may not be optimally matched to an associated antenna, resulting in poor sensitivity, reduced power output, or a reduced transmission efficiency, leading to wasted power. Wasted power, in turn, can result in a shortened battery life or a loss of communication link due to transmission failure (e.g., from the reduced power output, etc.).

In certain examples, a matching circuit can be used to reduce the impedance mismatch between the RF transceiver and the antenna. However, it can be difficult to provide an optimum match between the RF transceiver and the antenna, e.g., due to a complex or non-standard antenna, a varying dielectric medium surrounding the antenna before or after implantation, or a varying effective impedance changing with the surrounding environment.

In an example, a “stripline” measuring circuit on a printed circuit board including an antenna can be used to measure an impedance match between the antenna and an RF transceiver. However, the present inventors have recognized, among other things, that the “stripline” measuring circuit can consume a large amount of board space and can require a fairly large number of components to measure forward and reflected power.

This document discusses, among other things, an implantable telemetry system including an RF transceiver configured to drive an antenna and a matching circuit, configured to be coupled to the antenna, the matching circuit including a control parameter configured to adjust an impedance of the matching circuit. The implantable telemetry system also includes a control circuit configured to receive an indication of the RF transceiver current draw and to control the control parameter of the matching circuit using the received indication of current draw. In an example, the control parameter is controlled to decrease the RF transceiver current draw.

Example 1 describes subject matter that can include or use an implantable telemetry system that can include: a radio frequency (RF) transceiver configured to drive an antenna at a specified operating frequency; a matching circuit configured to be coupled to the antenna, the matching circuit having a control parameter configured to adjust the impedance of the matching circuit; and a control circuit configured to receive an indication of RF transceiver current draw and to control the control parameter using the received indication of current draw.

In Example 2, the subject matter of Example 1 can optionally include the control circuit being configured to vary the control parameter, to receive a plurality of indications of RF transceiver current draw at the different control parameter values, and to control the control parameter using the received plurality of indications of RF transceiver current draws.

In Example 3, the subject matter of any one of Examples 1 or 2 can optionally include the control circuit being configured to control the control parameter such that a control parameter setting corresponds to a minimum current draw for the RF transceiver.

In Example 4, the subject matter of any one of Examples 1-3 can optionally include the control circuit being configured to control the control parameter such that a control parameter setting corresponds to a current reading below a threshold current for the RF transceiver.

In Example 5, the subject matter of any one of Examples 1-4 can optionally include the control parameter setting corresponding to a current reading below a threshold current for the RF transceiver is an approximation of control parameter setting corresponding to a maximum power transfer from the RF transceiver to the antenna.

In Example 6, the subject matter of any one of Examples 1-5 can optionally include a current sensor coupled between a power supply and the RF transceiver for detecting the RF transceiver current draw.

In Example 7, the subject matter of any one of Examples 1-6 can optionally include the control circuit being configured to receive an indication of RF transceiver current draw while the RF transceiver is one of transmitting and receiving.

In Example 8, the subject matter of any one of Examples 1-7 can optionally include the control circuit being configured to periodically control the control parameter of the matching circuit.

In Example 9, the subject matter of any one of Examples 1-8 can optionally include the control circuit being configured to receive an external command having an indication to control the control parameter of the matching circuit, wherein the control circuit is configured to control the control parameter in response to the receiving the command.

In Example 10, the subject matter of any one of Examples 1-9 can optionally include the control circuit being configured to set the control parameter at a first setting for transmission and to set the control parameter at a second setting for reception.

Example 11 describes subject matter that can include, or can be combined with the subject matter of any one of Examples 1-10 to include: driving an antenna at a specified operating frequency with a radio frequency (RF) transceiver; receiving an indication of a current draw of the RF transceiver; controlling a control parameter of a matching circuit for the antenna using the detected current to decrease the RF transceiver current draw, wherein the control parameter is configured to adjust an impedance of the matching circuit.

In Example 12, the subject matter of any one of Examples 1-11 can optionally include: varying the control parameter of the matching circuit; detecting the current drawn by an RF transceiver of the implantable medical device as the parameter is varied to obtain a plurality of indications of the current draw of the RF transceiver; and wherein controlling includes controlling the control parameter using the plurality of indications of the current draw of the RF transceiver.

In Example 13, the subject matter of any one of Examples 1-12 can optionally include: determining a control parameter setting that corresponds to a minimum current draw for the RF transceiver; and wherein controlling includes controlling the control parameter of the matching circuit setting the control parameter to correspond to a minimum current draw for the RF transceiver.

In Example 14, the subject matter of any one of Examples 1-13 can optionally include: comparing the plurality of indications of the current drawn by the RF transceiver to a threshold current; and wherein controlling includes controlling the control parameter of the matching circuit to set the control parameter to correspond to a current reading below the threshold current for the RF transceiver.

In Example 15, the subject matter of any one of Examples 1-14 can optionally include the current reading below the threshold current for the RF transceiver being an approximation of a maximum power transfer from the RF transceiver to the antenna.

In Example 16, the subject matter of any one of Examples 1-15 can optionally include the detecting the current draw of the RF transceiver including detecting while the RF transceiver is one of transmitting and receiving.

In Example 17, the subject matter of any one of Examples 1-16 can optionally include the controlling including controlling the control parameter periodically.

In Example 18, the subject matter of any one of Examples 1-17 can optionally include: receiving a command to control the control parameter of the matching circuit; and wherein controlling includes controlling the control parameter in response to the command.

Example 19 describes subject matter that can include, or that can be combined with the subject matter of any one of Examples 1-18 to optionally include or use an antenna; a matching circuit coupled to the antenna, the matching circuit configured to vary a control parameter of the matching circuit; a radio frequency (RF) transceiver coupled to the matching circuit; a power supply coupled to the RF transceiver; a current sensor configured to detect a current between the power supply and the RF transceiver as the matching circuit varies the control parameter across a range of values; a control circuit configured to compare the detected current to a current threshold; and wherein the control circuit is configured to set the control parameter to a setting that corresponds to a current draw below the current threshold.

In Example 20, the subject matter of any one of Examples 1-19 can optionally include the control parameter including an impedance of the matching circuit.

These examples can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally an example schematic of an implantable telemetry system.

FIG. 2 illustrates generally an example of a method for controlling an impedance of a matching circuit using a current draw of an RF transceiver.

FIG. 3 illustrates generally a graph of example curves showing RF transceiver current draw and power transfer from an RF transceiver to an antenna versus a control parameter setting.

### DETAILED DESCRIPTION

The present inventors have realized, among other things, that the current draw of the RF transceiver provides an indication of the quality of the match between an RF transceiver and an antenna. In an example, when the antenna is mismatched (e.g., improperly tuned) with the RF transceiver, the current draw of the RF transceiver can be used to approximate an impedance setting for the matching circuit that maximizes the power transfer to/from the antenna and minimizes the current draw of the RF transceiver.

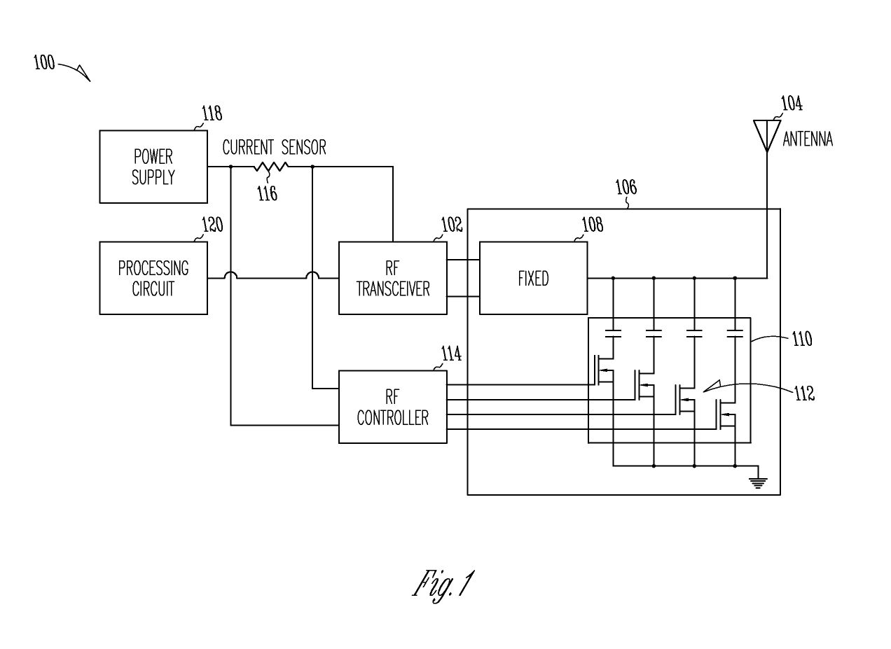

FIG. 1 illustrates generally an example of a schematic for an implantable telemetry system 100 that can be coupled to an IMD (e.g. a pacemaker). The implantable telemetry system 100 of FIG. 1 can include an RF transceiver 102 coupled to an antenna 104 for radiating and receiving signals for the RF transceiver 102. The RF transceiver 102 is configured to drive the antenna 104 at specified frequency for transmission and reception of signals.

The implantable telemetry system 100 of FIG. 1 can include a power supply 118 coupled to the RF transceiver 102 to provide power for operation of the RF transceiver 102. The RF transceiver 102 can also be coupled to a processing circuit 120 that can provide information to the RF transceiver 102 for transmission, and can process information received by the RF transceiver 102.

The implantable telemetry system of FIG. 1 can include a matching circuit 106 coupled between the RF transceiver 102 and the antenna 104 for matching the impedance of the RF transceiver 102 to the antenna. In an example, matching the impedance can include setting the impedance of the RF transceiver 102 to have the complex conjugate of the impedance of the antenna 104. Changing the impedance of the matching circuit 106 changes the total impedance seen by the antenna 104, since the matching circuit 106 is coupled between the RF transceiver 102 and the antenna 104. When the impedance of the RF transceiver 102 is the complex conjugate of the impedance of the antenna 104, the reactive components of the impedances cancel out.

In an example, at least one control parameter in the matching circuit 114 can be controlled to adjust the impedance of the matching circuit 106 to match the impedance of the antenna 104 to the RF transceiver 102. In certain examples, the matching circuit 106 can include a fixed portion 108 and an adjustable portion 110, and the control parameter can be configured to change the configuration of the adjustable portion 110 to vary the impedance of the matching circuit 106. In an example, the control parameter can change which of a plurality of capacitors 112 in the adjustable portion 110 are coupled to a ground potential. The configuration of the plurality of capacitors 112 can establish an impedance value for the adjustable portion 110. In other examples, inductors or other circuit elements can be used as control parameters in place of, or in addition to the plurality of capacitors 112.

The implantable telemetry system of FIG. 1 can include a control circuit 114 for controlling the adjustable portion 110 of the matching circuit 106. In an example, the control circuit 114 includes digital circuitry. The control circuit 114 can control the adjustable portion 110 using information obtained by a current sensor 116. The current sensor 116 can be coupled between the power supply 118 and the RF transceiver 102. The current sensor 116 can detect the current draw of the RF transceiver 102 and provide readings indicative of the current draw to the control circuit 114. In an example, the current sensor 116 can include a resistor coupled in series between the power supply 118 and the RF transceiver 102. The current sensor 116 can also include an analog to digital converter (ADC) for converting current values sensed by the resistor to digital readings for the control circuit 114. In another example, the control circuit 114 can obtain the voltage values across the resistor directly for use as current draw readings (e.g., current=voltage/resistance).

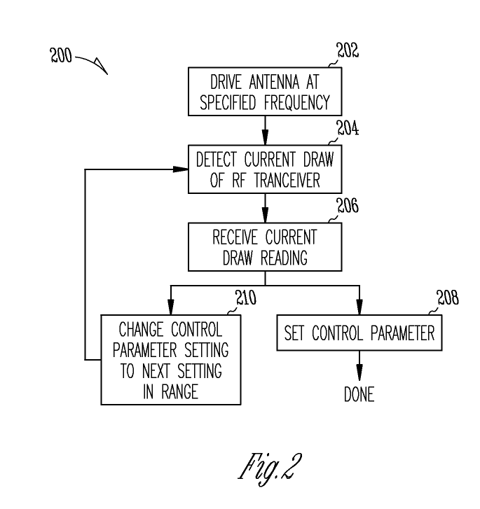

FIG. 2 illustrates generally an example of a method 200 for adjusting the impedance of the matching circuit 106 using a current draw of the RF transceiver 102. At block 202, the RF transceiver 102 can drive the antenna 104 at a specified operating frequency for radiation of signals therefrom. In an example, the RF transceiver 102 drives the antenna in the ultra-high frequency (UHF) range. In an example, the RF transceiver 102 drives the antenna at 870 MHz. In other examples, the RF transceiver 102 can drive the antenna at one or more other frequencies.

At block 204, an indication of the current draw of the RF transceiver 102 is obtained. In an example, the indication of the current draw can be obtained by a current sensor 116. In an example, the current sensor 116 can detect the current draw while the RF transceiver 102 is transmitting. In another example, the current sensor 116 can detect the current draw while the RF transceiver 102 is receiving. In yet another example, the current sensor 116 can detect the current draw at least once while the RF transceiver 102 is transmitting and at least one other time while the RF transceiver 102 is receiving. Thus, the current sensor 116 can obtain separate readings for transmission and for reception. The one or more readings obtained by the current sensor 116 can be provided to the control circuit 114.

In an example, the indication of the current draw of the RF transceiver 102 can be obtained with a coulomb counter. The coulomb counter can measure the quantity of charge drawn by the RF transceiver 102. The quantity of charge can be used as an indication of the current drawn. The readings measured by the coulomb counter can be provided to the control circuit 114.

At block 206, the control circuit 114 can receive the one or more readings as an indication of the current draw of the RF transceiver 102. As mentioned above, in one example, the reading can include a digital value. At block 208, the control circuit 114 can control a control parameter for the matching circuit 106 using the current draw reading from the current sensor 116. In an example, the control circuit 114 can control the control parameter by changing a setting of the control parameter to adjust the impedance of the matching circuit 106. Each setting of the control parameter can correspond to a configuration of the plurality of capacitors 112. In another example, the control circuit 114 can control the control parameter by holding the control parameter at a present setting to maintain the impedance of the matching circuit 106 at a present level.

In an example, at block 210, the control parameter of the matching circuit 106 can be changed after a current draw reading is obtained, and another current draw reading is obtained by the current sensor 116. The loop of changing the control parameter and obtaining a reading can be repeated such that the control parameter is varied across a range of values and a current draw reading is obtained at each control parameter value. Accordingly, in an example, a plurality of current draw readings are obtained which correspond to a plurality of control parameter settings. In an example, the control parameter can be varied by stepping the control parameter through a series of values. In an example, the control parameter can be varied across a large range of values at a first time and at a second time the control parameter is varied across a narrow range of values. The large range of values can, for example, be for “coarse” tuning and the narrow range of values can be for fine tuning.

In an example, after the control parameter has been varied across the range of values and the corresponding current draw readings have been obtained, the control circuit 114 can set the control parameter using the plurality of current draw readings. In an example, the control circuit 114 can set the control parameter to correspond to a current reading that is below a threshold current for the RF transceiver 102. Setting the control parameter to below the threshold current can also sets the RF transceiver to transmit using a magnitude of current below the threshold current; notably, the RF transceiver can transmit with the magnitude of current corresponding to the control parameter setting. Thus, the plurality of current draw readings can be used to control the magnitude of current used by the RF transceiver 102. In an example, the control circuit 114 can set the control parameter at a first setting for transmission and at a second setting for reception. The first setting can be determined based on current draw readings detected while the RF transceiver 102 is transmitting Likewise, the second setting can be determined based on current draw reading detected while the RF transceiver 102 is receiving.

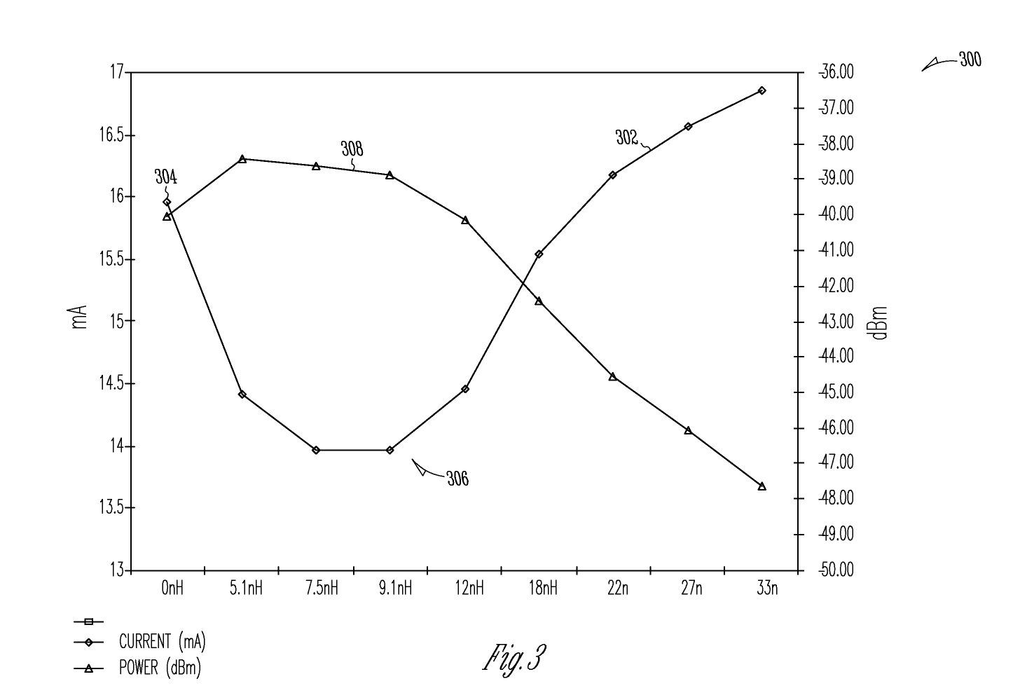

FIG. 3 illustrates a graph 300 of an example curve 302 showing current draw readings obtained across a range of control parameter settings. In an example, the graph 300 shows mA of current draw for the RF transceiver 102 on the vertical axis and a control parameter setting (nH of inductance for variable inductance) of the matching circuit 106 on the horizontal axis. In an example, the curve 302 illustrates the current draw when the antenna 104 is mismatched to the RF transceiver 102 (e.g., when the antenna 104 is not properly tuned to the specified frequency that the RF transceiver 102 is driving the antenna 104).

The curve 302 can include a peak 304 followed by a dip 306 in the current readings as the impedance of the matching circuit 106 increases. After the dip 306, the current drawn by the RF transceiver rises again. The dip 306 in the current readings corresponds to improved matching between the RF transceiver 102 and the antenna 104.

The improvement in matching between the RF transceiver 102 and the antenna 104 can also improve the power transferred from the RF transceiver 102 to the antenna 104. The transferred power (dBm) is shown in curve 308 of graph 300 (vertical axis) versus the control parameter settings (horizontal axis). As shown by curve 308, the power transferred is high when the current draw is low (dip 306) which again corresponds to improved matching between the RF transceiver 102 and the antenna 104. The power transferred decreases as the current draw increases and the match between the RF transceiver 102 and the antenna 104 decreases.

In an example, a threshold current draw can be selected and the control parameter can be set to correspond to a current draw below the threshold. In an example, the threshold current draw can be selected as 15 mA. Thus, in curve 302 any of the settings corresponding to a current draw below 15 mA can be selected as the setting for the control parameter. Accordingly, the current draw for the RF transceiver 102 can be reduced or maintained below a desired magnitude. Additionally, good power transfer to and from the antenna 104 can be achieved without directly measuring the output power from the antenna 104, since any control parameter setting corresponding to a current draw below 15 mA can be used as an approximation of a good power transfer to and from the antenna 104.

In an example, the control circuit 114 can set the control parameter to correspond to the minimum current draw for the RF transceiver 102 across the range of control parameter settings. Accordingly, this can enable the RF transceiver 102 to use a low current draw, and in certain examples, the least current draw possible.

In other examples, the current sensor 116 can be coupled between the power supply and a power amplifier for the RF transceiver 102 or another (or the same) current sensor 116 can be coupled between the power supply and a low noise amplifier (LNA) for the RF transceiver 102. In these examples, the current sensor 116 can detect the current draw of the power amplifier or the LNA directly. The power amplifier and the LNA for the RF transceiver 102 are the components whose current draw changes in conjunction with the control parameter setting for the matching circuit 106. Accordingly, in an example, the current sensor 116 can detect the current draw of these components directly. However, in many applications it can be difficult to isolate the current draw for the power amplifier and the LNA. Accordingly, (as described above) the current draw for the RF transceiver 102 as a whole while transmitting can be used to approximate the current draw of the power amplifier and the LNA since these components represent a large percentage of the current draw for the RF transceiver 102, and the current draw by most other components is static.

In an example, the control circuit 114 can periodically control the control parameter setting. Accordingly, on a periodic basis the control parameter is varied across a range of values and a plurality of current draw levels are obtained. A control parameter setting is then selected by the control circuit 114 using the plurality of current draw levels.

In an example, the control circuit 114 can control the control parameter setting in response to an external command. Thus, for example, a programmer can cause the implantable telemetry system 100 of FIG. 1 to control the control parameter when the environment surrounding the implantable telemetry system 100 changes. Environmental changes can occur when the implantable telemetry system 100 is moved from external to a subject to internal (implanted) to the subject. Additionally, the control circuit 114 can control the control parameter when a transmit/receive frequency for the RF transceiver changes. In another example, the control circuit 114 can be programmed to automatically control the control parameter when the transmit/receive frequency for the RF transceiver changes.

To control the control parameter using an external command, the RF transceiver 102 can receive an external command and send the received command to the processing circuit 120 for processing. The command can be provided to the control circuit 114 to control the parameter setting. The control parameter can be varied across a range of values and a plurality of current draw levels are obtained. A control parameter setting can then be selected by the control circuit 114 using the plurality of current draw levels.

In an example, the control circuit 114 can be programmed to control the control parameter in frequent intervals (e.g. once every 10 minutes) during the first period of operation after the start of the IMD (e.g. the first 10 hours of operation) including the implantable telemetry system 100, and then control the control parameter in longer intervals (e.g. once every month) after the first period of operation. Thus, the implantable telemetry system 100 can be adjusted frequently soon after it is turned on to account for possible drastic changes in surrounding environment as the associated IMD is implanted. Then after the implantable telemetry system is implanted the control parameter can be updated less frequently to preserve battery life.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile tangible computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An implantable telemetry system comprising:
a radio frequency (RF) transceiver configured to drive an antenna at a specified operating frequency;
a matching circuit configured to be coupled to the antenna, the matching circuit having a control parameter configured to adjust the impedance of the matching circuit; and
a control circuit configured to receive an indication of RF transceiver current draw and to control the control parameter using the received indication of current draw.

2. The implantable telemetry system of claim 1, wherein the control circuit is configured to vary the control parameter, to receive a plurality of indications of RF transceiver current draw at the different control parameter values, and to control the control parameter using the received plurality of indications of RF transceiver current draws.

3. The implantable telemetry system of claim 2, wherein the control circuit is configured to control the control parameter such that a control parameter setting corresponds to a minimum current draw for the RF transceiver.

4. The implantable telemetry system of claim 2, wherein the control circuit is configured to control the control parameter such that a control parameter setting corresponds to a current reading below a threshold current for the RF transceiver.

5. The implantable telemetry system of claim 4, wherein the control parameter setting corresponding to a current reading below a threshold current for the RF transceiver is an approximation of control parameter setting corresponding to a maximum power transfer from the RF transceiver to the antenna.

6. The implantable telemetry system of claim 1, comprising a current sensor coupled between a power supply and the RF transceiver for detecting the RF transceiver current draw.

7. The implantable telemetry system of claim 1, wherein the control circuit is configured to receive an indication of RF transceiver current draw while the RF transceiver is one of transmitting and receiving.

8. The implantable telemetry system of claim 1, wherein the control circuit is configured to periodically control the control parameter of the matching circuit.

9. The implantable telemetry system of claim 1, wherein the control circuit is configured to receive an external command having an indication to control the control parameter of the matching circuit, wherein the control circuit is configured to control the control parameter in response to the receiving the command.

10. The implantable telemetry system of claim 1, wherein the control circuit is configured to set the control parameter at a first setting for transmission and to set the control parameter at a second setting for reception.

11. A method comprising:
driving an antenna at a specified operating frequency with a radio frequency (RF) transceiver;
receiving an indication of a current draw of the RF transceiver;
controlling a control parameter of a matching circuit for the antenna using the detected current to decrease the RF transceiver current draw, wherein the control parameter is configured to adjust an impedance of the matching circuit.

12. The method of claim 11, comprising:
varying the control parameter of the matching circuit;
detecting the current drawn by an RF transceiver of the implantable medical device as the parameter is varied to obtain a plurality of indications of the current draw of the RF transceiver; and
wherein controlling includes controlling the control parameter using the plurality of indications of the current draw of the RF transceiver.

13. The method of claim 12, comprising:
determining a control parameter setting that corresponds to a minimum current draw for the RF transceiver; and
wherein controlling includes controlling the control parameter of the matching circuit setting the control parameter to correspond to a minimum current draw for the RF transceiver.

14. The method of claim 12, comprising:
comparing the plurality of indications of the current drawn by the RF transceiver to a threshold current; and
wherein controlling includes controlling the control parameter of the matching circuit to set the control parameter to correspond to a current reading below the threshold current for the RF transceiver.

15. The method of claim 13, wherein a current reading below the threshold current for the RF transceiver is an approximation of a maximum power transfer from the RF transceiver to the antenna.

16. The method of claim 11, wherein detecting the current draw of the RF transceiver includes detecting while the RF transceiver is one of transmitting and receiving.

17. The method of claim 11, wherein controlling includes controlling the control parameter periodically.

18. The method of claim 11, comprising:
receiving a command to control the control parameter of the matching circuit; and
wherein controlling includes controlling the control parameter in response to the command.

19. An implantable medical device comprising:
an antenna;
a matching circuit coupled to the antenna, the matching circuit configured to vary a control parameter of the matching circuit;
a radio frequency (RF) transceiver coupled to the matching circuit;
a power supply coupled to the RF transceiver;
a current sensor configured to detect a current between the power supply and the RF transceiver as the matching circuit varies the control parameter across a range of values;
a control circuit configured to compare the detected current to a current threshold; and
wherein the control circuit is configured to set the control parameter to a setting that corresponds to a current draw below the current threshold.

20. The implantable medical device of claim 19, wherein the control parameter includes an impedance of the matching circuit.
